(19) United States
(12) Patent Application Publication
(10) Pub. No.: US 2003/0125659 A1
(43) Pub. Date: Jul. 3, 2003

(54) Dosage of transdermal delivery systems

(76) Inventor: Wolfgang Fleischer, Ingelheim (DE)

Correspondence Address: PENNIE & EDMONDS LLP, 1667 K STREET NW, SUITE 1000, WASHINGTON, DC, 20006

(21) Appl. No.: 10/302,970

(22) Filed: Nov. 25, 2002

Related U.S. Application Data
(63) Continuation-in-part of application No. PCT/EP01/06008, filed on May 25, 2001.

(30) Foreign Application Priority Data
May 25, 2000 (DE) 100 25 890.5

(51) Int. Cl. A61N 1/30

(52) U.S. Cl. 604/19

(57) ABSTRACT

A portable device for dispensing a length of a matrix-controlled transdermal delivery system having a dose of active agent having a carriage device for supporting a first length of matrix-controlled transdermal delivery system; a unit for entering a dose amount and transposing the dose amount into a second length of the matrix-controlled transdermal delivery system; a separating device for separating the second length of matrix-controlled transdermal delivery system from the first length of matrix-controlled transdermal delivery system; and a locking mechanism that can be activated or deactivated by entering a user code.

### 1. RELATED APPLICATIONS

The present application is a continuation-in-part of PCT Application No. PCT/EP01/06008, filed May 25, 2001, which published in English as WO 01/89490 on Nov. 29, 2001.

### 2. FIELD OF THE INVENTION

The invention relates to a process for the individual dosing of a matrix-controlled transdermal delivery system (also referred to hereinafter as matrix systems or transdermal therapeutic systems) as well as a dispenser that is suitable for such a process, and a matrix-controlled transdermal delivery device suitable for use in the device of the invention.

### 3. TECHNICAL FIELD

Non-invasive applications of substance preparations, which are suitable for penetrating a permeable barrier, such as the skin, are advantageous with many courses of therapy. Transdermal delivery systems have been in use for this purpose for about twenty years.

The epidermis constitutes a natural barrier against penetrating substances. Consequently, for transdermal applications, consideration is given to substances where only small quantities are necessary to provide a therapeutic effect; where transport through the skin occurs so that an adequate amount of the penetrating substance reaches the blood circulation (good water and lipid solubility); where long-term therapy is planned; and where skin tolerability is good.

Transdermal drug systems, also known as transdermal therapeutic systems or TTS, generally incorporate patches which vary in padding type, form, and size, which are adhered to areas of the skin and remain there, for up to several days. The active substance is stored in the patch and is discharged therefrom into the skin. For the delivery of the substance, it is necessary that the patch be in direct contact with the skin.

The best known active substance in TTS is glycerol trinitrate (GTN), which is used to treat coronary cardiac disease. When orally administered and because of its early breakdown due to its pronounced “first pass” effect, it only reaches the targeted organ to a minor degree. The second most common therapy target focuses on people giving up smoking and includes several nicotine TTS with which a constant nicotine plasma level is achieved. Also, on the market are TTS that continuously discharge synthetic opiates for the treatment of chronic pain. Here, too, a constant plasma level is achieved that lasts for several days.

The delivery of the drug substance takes place under controlled permeation conditions from a reservoir through a membrane or by diffusion from a matrix. Accordingly, TTS patches have been classified into two main groups: reservoir systems (membrane-controlled TTS) and matrix-controlled TTS.

With reservoir systems, the patch comprises, from the outside inwards, a skin-colored, impermeable covering sheet (support carrier coating), a semi-solid substance suspension in silicon oil or polyisobutylene (reservoir), a control polymer membrane, an adhesive layer (adhesive coating) with the initial dose, and strip-off sheeting. The delivery of the substance is continuous and is controlled by the membrane that has a specific permeability for the substance in question. The disadvantage of the reservoir system is that if the membrane is damaged, the entire dose can be discharged at once (precipitated discharge).

Matrix-controlled TTS include systems which store the active agent in one or more polymer layers which may include or consist of the adhesive layer. Matrix-controlled TTS generally contain a homogenous dispersion of solid and dissolved substance particles in a lipophilic or hydrophilic polymer matrix mainly made up of an acryl resin or an acrylic resin as a drug reservoir. Delivery is controlled by the polymer matrix and/or the adhesive layer. Many matrix patches can be cut up, for example with a pair of scissors. Matrix systems do not discharge the active substance in a steady state. Precipitated discharge of the entire dose, as with membrane systems, is not possible, for which reason these matrix-controlled TTS are considered safer for highly potent pharmaceutical substances.

In modem matrix patches, control mechanisms have been developed to assure a practically constant delivery of the pharmaceutical substance. With depot-effect patches in the form of a mono-layered matrix, the substance is bound in a hydrophilic polymer matrix that contains water, in particular a gel system. In this case, delivery depends on a defined swelling performance of the matrix. With systems in the form of a multi-layered matrix (for example formed by multi-layered laminas) the pharmaceutical substance is fixed depot-like to a carrier such as lactose, and embedded, partially dissolved, in a self-adhesive resin compound. With micro-reservoir systems, a silicon elastomer matrix contains many substance-bearing micro-compartments, on the order of 10 to 200 μm in size. The substance is delivered from these microparticles by controlled dissolution.

Compared to conventional pharmaceutical forms, a TTS has the following advantages:

1) Avoids initially passing through the liver (averts a “first pass” effect).

2) Level of active substance is largely constant and hence no underdose or overdose of substances with a narrow therapeutic spectrum of required effect.

3) Improvement of application compliance because the effect lasts several days with fewer side-effects.

4) With incompatibility reactions or when no longer required, the action can be interrupted by removal of the patch.

5) No gastro-intestinal incompatibility reactions.

With the matrix-controlled transdermal delivery system, the substance is applied either directly on the underside of the patch and diffused over the entire surface of the patch into the biological system, or is mixed with the adhesive agent, i.e., with an adhesive, and diffuses from this adhesive agent into the biological system. Whereas with a reservoir system, the overall dose of substance is specified by the appropriately defined volume of the reservoir, with matrix-controlled transdermal delivery systems, the quantity of the diffused substance can be determined by the size of the applied matrix, i.e., patch. With an even layering of the patch, the dose of the active substance is proportional to the area of the patch.

Conventional transdermal patches are usually packed singly in sealed bags. The user instructions that typically accompany the patch particularly relates to the dose of the substance or substances and to the dose that is to be delivered within a certain period of time. The conventional TTS details a defined size and hence an appropriate dose. With transdermal systems that have a matrix formulation, the dose is equivalent to the given size, for example in cm², of the patch. The patch is usually coated in whole and apportioned into sections with a suitable cutting tool, and is then packaged. Consequently, different package sizes provide different strengths of action.

A disadvantage with conventional transdermal therapeutic systems available on the market is that they give specified dosages which only provide approximations to the individual patients' requirements—a fact which is true, for example, for tablets, suppositories, and other oral application forms as well. It is not infrequent for dosage steps to be at intervals of 50 to 100%. It would hence be desirable if the active substance or substances of transdermal therapeutic systems could be individually and precisely adjusted to a specific dose for a specific individual for a specific therapeutic purpose, such as is possible for example, with injectable agents and oral fluid forms whose amounts are varied for each individual dose, for example by specifying a numbers of drops.

There is a need in the art for a transdermal therapeutic system whose dosage can be fine-tuned and tailored for an individual in need of treatment. There is a further need for a device that permits a matrix-controlled transdermal delivery system to be easily measured and administered to a patient. There is also a need for such a device that prevents unauthorized individuals from accessing the therapeutic agents located within the device. There is also a need for such a device that prevents authorized individuals from receiving more than the prescribed dose. The present invention discloses a method and device that addresses these needs.

### 4. SUMMARY OF THE INVENTION

The present invention is directed to a portable device for dispensing a length of a matrix-controlled transdermal delivery system having a dose of active agent comprising: (i) a carriage device for supporting a first length of matrix-controlled transdermal delivery system; (ii) a unit for entering a dose amount and transposing the dose amount into a second length of the matrix-controlled transdermal delivery system; (iii) a separating device for separating the second length of matrix-controlled transdermal delivery system from the first length of matrix-controlled transdermal delivery system; and (iv) a locking mechanism that can be activated or deactivated by entering a user code.

In one embodiment, the unit for entering the dose continuously sets the dose within a specified range. In another embodiment, the matrix-controlled transdermal delivery system is a patch evenly coated with the active agent. In another embodiment, the active agent is directly applied to a surface of the patch. In another embodiment, the active agent is combined with an adhesive or adhesive matrix.

In one embodiment, the matrix-controlled transdermal delivery system is a mono-layered matrix. In another embodiment, the matrix-controlled transdermal delivery system is a multi-layered matrix. In another embodiment, the active agent is selected from glycerol trinitrate, nicotine, an opiate, an opioid, and mixtures thereof. In another embodiment, the active agent is an opioid or an opiate. In another embodiment, the unit for entering the dose is a mechanical unit. In another embodiment, the unit for entering the dose is an electronic unit.

In one embodiment, the device further comprises a printing unit for providing a delivery slip. In another embodiment, the device further comprises a calculating system for determining the amount of active agent dispensed over a period of time. In yet another embodiment, the matrix-controlled transdermal delivery system is maintained and dispensed under sterile conditions.

The present invention is also directed to a process for preparing a length of a matrix-controlled transdermal delivery system having a dose of an active agent comprising the steps of (i) applying a second length of matrix-controlled transdermal delivery system comprising an active agent on a carriage means; (ii) deactivating a locking mechanism; (iii) specifying a dose; (iv) transposing the dose into a first length of the matrix-controlled transdermal delivery system; (v) separating the first length of the matrix-controlled transdermal delivery system; (vi) optionally packaging the first length of the matrix-controlled transdermal delivery system; (vii) optionally providing a delivery slip; and (viii) optionally calculating the amount of active agent contained within the matrix-controlled transdermal delivery system over a period of time.

In one embodiment, the matrix-controlled transdermal delivery system is a patch coated with the active agent. In another embodiment, the active agent is applied to a surface of the patch. In another embodiment, the active agent is combined with an adhesive or adhesive matrix. In another embodiment, the transposing of the dose into the first length of matrix-controlled transdermal delivery system is effected mechanically. In another embodiment, the transposing of the dose into the first length of the matrix-controlled transdermal delivery system is effected electronically.

The present invention is also directed to a matrix-controlled transdermal delivery system comprising an active agent, having a length, and having markings provided at intervals of the length.

In one embodiment, the markings are an imprint or a perforation. In another embodiment, the markings can be read by a portable device for dispensing the matrix-controlled transdermal delivery system.

In one embodiment the matrix-controlled transdermal delivery system is in the form of a patch coated with active agent. In another embodiment, the agent is applied to surface of the patch. In another embodiment, the active agent is combined with an adhesive or an adhesive matrix.

In one embodiment, the matrix-controlled transdermal delivery system is in the form of a mono-layered matrix. In another embodiment, the matrix-controlled transdermal delivery system is in the form of a multi-layered matrix.

In one embodiment, the active agent is selected from glycerol trinitrate, nicotine, an opiate, or an opioid. In another embodiment, the active agent is an opiate, an opioid, and mixtures thereof. In another embodiment, the matrix-controlled transdermal delivery system further comprises a penetration enhancer.

### 5. BRIEF DESCRIPTION OF THE DRAWINGS

FIG. 1 shows a simplified representation of an exemplary embodiment of a device for individual dosage and dispensing of matrix-controlled transdermal delivery systems according to the present invention.

FIG. 2 shows an exemplary embodiment of a matrix-controlled transdermal delivery system according to the present invention.

FIG. 3 shows another exemplary embodiment of a matrix-controlled transdermal delivery system according to the present invention.

### 6. DESCRIPTION OF PREFERRED EMBODIMENTS

The invention attains the objects of the present invention by providing a dispensing device for the individual dosage and dispensing of a length of a matrix-controlled transdermal delivery system, which comprises a matrix-controlled transdermal delivery system of specified width provided with at least one pharmaceutically active agent, a carriage means for the matrix-controlled transdermal delivery system and a separating device for the matrix-controlled transdermal delivery system. A pertinent feature of the device relates to a unit appliance i.e., a module, that specifies the dosage of the substance and serves to convert a required dosage into an appropriate length of the matrix-controlled transdermal delivery system that is to be dispensed. Hence, said unit appliance enables an individual dosage of the substance to be administered by means of separating a length of the matrix-controlled transdermal delivery system corresponding to the desired dosage, using the separating device. In one embodiment, the unit appliance for specifying the substance dosage enables continuous selection of the required dosage within the adjustment range of the device as constituted by the invention.

Another particular feature of the device in accordance with the invention is that it can be made portable. Hence, the device of the invention can be used where it is required, for example in hospitals, pharmacies, doctors' offices and such for dispensing a length of a matrix-controlled transdermal therapeutic systems.

The device in accordance with the invention proves to be highly advantageous, since it enables individually adjusted dosages outside the fixed specified regular dose of a prefabricated transdermal patch, such as are regularly available on the market, to be dispensed to the patient. This is particularly advantageous when it is determined that an ideal dose for a patient receiving the transdermal patch lies between or outside of the range of the fixed dosage patches that are presently commercially available.

In one embodiment, the present invention provides matrix-controlled transdermal delivery systems, which are suitable for use in the above-described portable device for individual dosage and dispensing. The matrix-controlled transdermal delivery systems of the invention are marked at intervals to allow separation of a specified length corresponding to certain dose unit. By selecting the number of dose units, the substance amount can be precisely adjusted to the requirements of an individual patient. In order to effect adjustments as individual as possible, the markings are at short intervals. The marking is, for example, an imprint on the matrix-controlled transdermal delivery system or a perforation, so that the length of matrix-controlled transdermal delivery system can be separated at the required length. When the dispenser of the invention electronically relays dosage and discharge of the matrix-controlled transdermal delivery system, the markings are readable by the above-mentioned inventive device, so that the length of matrix-controlled transdermal delivery system is detached automatically at the marking.

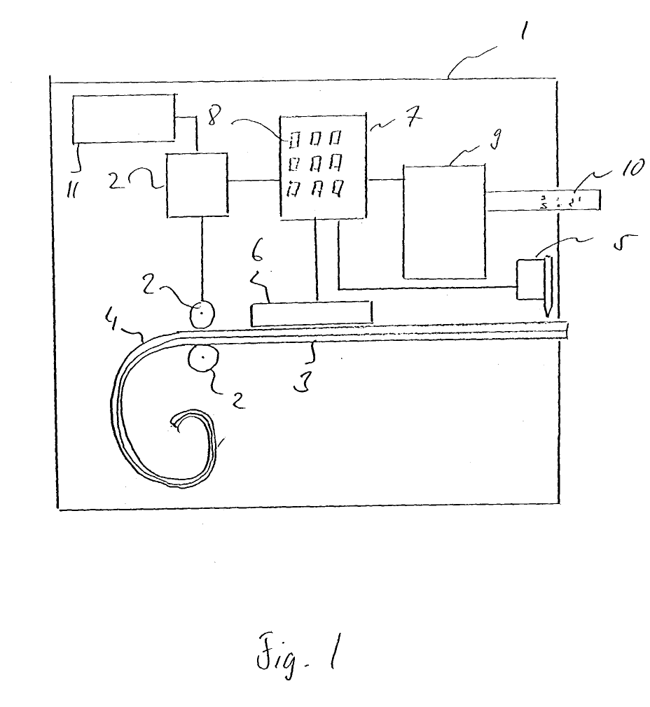

FIG. 1 shows an exemplary embodiment of a portable or stationary device for dispensing of a length of a matrix-controlled transdermal delivery systems 1 incorporating a matrix-controlled transdermal delivery system 4 of a specified width and coated with at least one active agent 3; a carriage means 2 for the matrix-controlled transdermal delivery system comprising for example a rotatable reel; a separating device 5 for separating a length of the matrix-controlled transdermal delivery system from the matrix-controlled transdermal delivery system, such as a cutting device; and a unit to specify the substance dose 6 which is a mechanical unit and/or an electronic unit that transposes a required dose into an appropriate length of the matrix-controlled transdermal delivery system to be dispensed. The device 1 further incorporates a locking mechanism 7 for preventing unauthorized operation of the device 1. The lock can be activated or deactivated by an individual user code input via a keyboard 8. The carriage means 2, the unit to specify the substance dose 6, the separating device 5, and the locking mechanism 7 are connected to each other and are connected to a printing unit 9 for producing a delivery slip 10 on which the quantity of substance delivered in the length of the matrix-controlled transdermal delivery system; the time of delivery, such as date and hour; the prescribing physician; and the user of the device, identified for example by the user code, may be recorded. Furthermore, a unit 11 for calculating the amount of substance dispensed over a specified period of time may be provided.

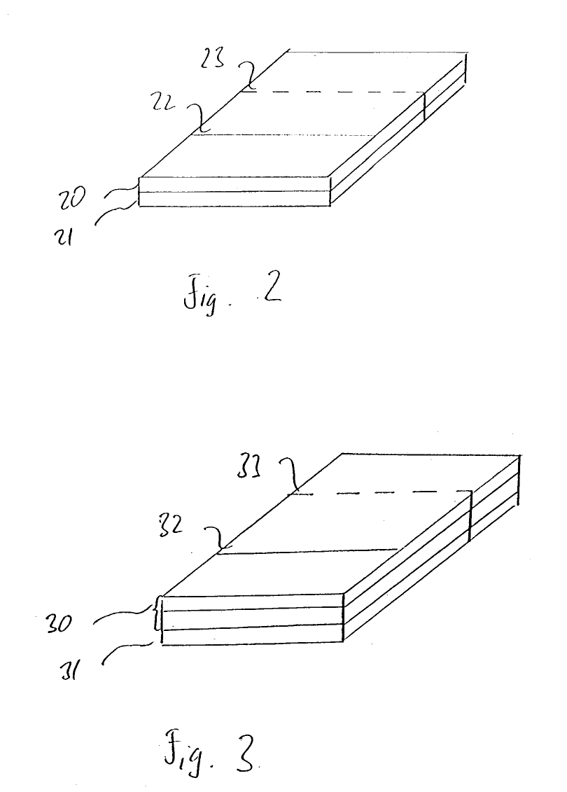

FIG. 2 shows an exemplary embodiment of a matrix-controlled transdermal delivery system having a matrix-controlled transdermal delivery system 20 and an active agent applied on the underside, wherein the matrix-controlled transdermal delivery system is provided in the form of a mono-layered matrix. The matrix-controlled transdermal delivery system shown in FIG. 2 is provided with markings 22 and 23 at intervals of length which correspond to particular dose units, the marking can be an imprint 22 or a perforation 23.

FIG. 3 shows another exemplary embodiment of a matrix-controlled transdermal delivery system according to the present invention, wherein a multilayered matrix 30 is covered with an active agent 31 on one side thereof. The matrix-controlled transdermal delivery system shown in FIG. 3 is provided with markings 32 and 33 at intervals of length which corresponds to particular dose units. The markings are an imprint 32 or a perforation 33.

### 6.1 Definitions

The phrases “transdermal delivery system” and “transdermal therapeutic system” as used herein means any device that when contacted with an animal's skin, can transdermally deliver a therapeutically effective amount of an active agent through the skin.

An animal includes, but is not limited to, a cow, monkey, horse, sheep, pig, chicken, turkey, quail, cat, dog, mouse, rat, rabbit, and guinea pig. In one embodiment an animal is a mammal. In another embodiment, an animal is a human.

The phrase “treatment of pain” or “treating pain” includes amelioration of pain or the cessation of pain in an animal.

The phrase “prevention of pain” or “preventing pain” includes the avoidance of the onset of pain in an animal.

The phrase “active agent” includes any substance used in treating pain or preventing pain. As used herein, any reference to any active agent includes all pharmaceutically acceptable forms of that agent, such as the salt form, the base form, the hydrate form, and the solvate form.

The phrase, “portable” means that the device can, for example, be operated as a hand-held device, or can be set up on an ordinary table.

### 6.2 Transdermal Delivery System

Any matrix-controlled transdermal delivery system known to those skilled in the art for transdermally delivering an active agent to an animal can be used for the transdermal-delivery device of the invention (See, e.g., H. S. Tan et al., Pressure Sensitive Adhesives for Transdermal Drug Delivery Systems, in PSTT 2(2):60-79 (1999), the disclosure of which is incorporated herein by reference). The transdermal-delivery device is designed so that when contacted with the animal's skin, a therapeutically effective amount of active agent is transdermally administered to the animal. Matrix-controlled delivery systems include systems which store the active agent in one or more polymer layers which may include or consist of the adhesive layer.

In one embodiment, the matrix-controlled transdermal delivery system may be a polymer-matrix design. In the matrix design, the active agent is dispersed in a matrix that partially controls the delivery rate of the active agent. The matrix reservoir is supported on an impermeable backing layer. A release liner protects the adhesive surface and the surface of the matrix. To administer an active agent, the release liner is removed to expose the matrix and the pressure-sensitive adhesive, and the device is contacted with the skin. When the matrix is contacted with the skin, an active agent diffuses out of the matrix, contacts the animal's skin, and penetrates the skin. The delivery rate is such that a therapeutically effective amount of an active agent is delivered to the animal.

In another embodiment, the matrix-controlled transdermal delivery system may be a drug-in-adhesive type transdermal delivery system. The drug-in-adhesive type transdermal delivery device comprises an active agent dispersed directly in a pressure-sensitive adhesive matrix. The adhesive matrix is typically supported on the topside with an impermeable backing film. To administer an active agent, the release liner is removed to expose the adhesive matrix, and the device is contacted with the skin. The adhesive matrix functions to adhere the device to the skin and, typically, to control the delivery rate of an active agent. The drug-in-adhesive design allows an active agent to diffuse out of the adhesive matrix, contact the animal's skin, and penetrate the skin. The delivery rate of an active agent is partially determined by the rate of diffusion of the active agent out of the adhesive matrix. The delivery rate is such that an therapeutically effective amount of an active agent is delivered to the animal.

The matrix-controlled transdermal delivery system is well-known to those skilled in the art (See, e.g., H. S. Tan et al., Pressure Sensitive adhesives for Transdermal Drug Delivery Systems, in PSTT 2(2):60-79 (1999)), the contents of which are expressly incorporated herein by reference).

The backing layer can be any suitable material that is impermeable to the contents of the reservoir compartment, the polymer matrix, or the adhesive matrix. Suitable materials for backing films are well known to those skilled in the art and include, but are not limited to, occlusive polymers such as polyurethane, polyesters such as poly(ethylene phthalate), polyether amide, copolyester, polyisobutylene, polyesters, high and low density polyethylene, polypropylene, polyvinylchloride, metal foils, and metal foil laminates of suitable polymer films.

Suitable materials for the matrix are well known to those skilled in the art and include, but are not limited to, polyethylene; polypropylene; ethylene/propylene copolymers; ethylene/ethylacrylate copolymers; ethylene/vinyl acetate copolymers; silicone elastomers, especially the medical-grade polydimethylsiloxanes; neoprene rubber; polyisobutylene; chlorinated polyethylene; polyvinyl chloride; vinyl chloride-vinyl acetate copolymer; polymethacrylate polymer (hydrogel); polyvinylidene chloride; poly(ethylene terephthalate); butyl rubber; epichlorohydrin rubbers; ethylene-vinyl alcohol copolymer; ethylene-vinyloxyethanol copolymer; silicone copolymers, for example, polysiloxane-polycarbonate copolymers, polysiloxane-polyethyleneoxide copolymers, polysiloxane-polymethacrylate copolymers, polysiloxane-alkylene copolymers (e.g., polysiloxane-ethylene copolymers), polysiloxane-alkylenesilane copolymers (e.g., polysiloxaneethylenesilane copolymers), and the like; cellulose polymers, for example methyl or ethyl cellulose, hydroxypropyl methyl cellulose, and cellulose esters; polycarbonates; polytetrafluoroethylene; and combinations thereof. In one embodiment, the matrix has a glass-transition temperature below room temperature. The polymer can, but need not necessarily, have a degree of crystallinity at room temperature. Cross-linking monomeric units or sites can be incorporated into the polymers. For example, cross-linking monomers can be incorporated into polyacrylate polymers. Known cross-linking monomers for polyacrylate polymers include, but are not limited to, polymethacrylic esters of polyols such as butylene diacrylate and dimethacrylate, trimethylol propane trimethacrylate, and the like. Other monomers that provide cross-linking sites include allyl acrylate, allyl methacrylate, diallyl maleate, and the like.

Suitable materials for the pressure-sensitive adhesive matrix are well known to those skilled in the art and include, but are not limited to, polyisobutylenes, polysiloxanes, and polyacrylate copolymers (polyacrylic esters), natural rubber/karaya gum-based adhesives, hydrogels, hydrophilic polymers, and polyurethanes such as those described in H. S. Tan et al., Pressure Sensitive Adhesives for Transdermal Drug Delivery Systems, in PSTT 2(2):60-79 (1999), the disclosure of which is incorporated herein by reference. The adhesive may further comprise modifying monomers, tackifiers, plasticizers, fillers, waxes, oils, and other additives to impart the desired adhesive properties. Id. In addition, one skilled in the art can readily achieve desired adhesive properties by the incorporation of materials such as initiators, crosslinkers and comonomers.

The matrix-controlled transdermal delivery system can optionally comprise one or more penetration enhancers, which increase the rate at which an active agent penetrates through the animal's skin. In another embodiment, the penetration enhancer penetrates the rate-controlling membrane or diffuses out of the polymer matrix or adhesive matrix so that it can contact the animal's skin and improve penetration of an active agent through the animal's skin. Suitable penetration enhancers for use in the transdermal-delivery devices and methods of the invention include, but are not limited, C₂-C₄ alcohols such as ethanol and isopropanol, polyethylene glycol monolaurate, diethyl glycol monomethyl ether, polyethylene glycol-3-lauramide, dimethyl lauramide, dimethyl isosorbide, sorbitan trioleate, fatty acids, esters of fatty acids having from about 10 to about 20 carbon atoms, monoglycerides or mixtures of monoglycerides of fatty acids having a total monoesters content of at least about 51% where the monoesters are those with from 10 to 20 carbon atoms, and mixtures of mono-, di- and tri-glycerides of fatty acids. Suitable fatty acids include, but are not limited to lauric acid, myristic acid, stearic acid, oleic acid, linoleic acid and palmitic acid. Monoglyceride permeation enhancers include glycerol monooleate, glycerol monolaurate, and glycerol monolinoleate, for example. Examples of penetration enhancers useful in the methods of the invention include, but are not limited to those described in U.S. Pat. Nos. 3,472,931; 3,527,864; 3,896,238; 3,903,256; 3,952,099; 3,989,816; 4,046,886; 4,130,643; 4,130,667; 4,299,826; 4,335,115; 4,343,798; 4,379,454; 4,405,616; 4,746,515; 4,316,893; 4,405,616; 4,060,084, 4,379,454; 4,560,553; 4,863,952; 4,863,970; 4,879,275; 4,940,586; 4,960,771; 4,973,468; 5,066,648; 5,164,406; 5,227,169; 5,229,130; 5,238,933; 5,308,625; 5,326,566; 5,378,730; 5,420,106; 5,641,504; 5,716,638; 5,750,137; 5,785,991; 5,837,289; 5,834,468; 5,882,676; 5,912,009; 5,952,000; 6,004,578; and Idson, J. Pharm. Sci. 64(b6):901-924 (1975), the disclosures of which are herein incorporated by reference.

The matrix-controlled transdermal delivery system can comprise a pharmacologically active agent that is capable of inducing a desired biological or pharmacological effect, which may include, but is not limited to, (1) affecting a living process; (2) having a prophylactic effect on an animal and preventing an undesired effect, such as preventing an infection; (3) alleviating a condition caused by, or a symptom of, a disease, e.g., pain or inflammation; and/or (4) alleviating, reducing, or eliminating a disease, condition, or symptom from the animal. The effect of the active agent may be local, such as for providing an anaesthetic effect, or it may be systemic or a combination thereof. General categories of active agents can, in one embodiment, include, but are not limited to: ACE inhibitors; adenohypophoseal hormones; adrenergic neuron blocking agents; adrenocortical steroids; inhibitors of the biosynthesis of adrenocortical steroids; alpha-adrenergic agonists; alpha-adrenergic antagonists; selective alpha-two-adrenergic agonists; androgens; anti-addictive agents; antiandrogens; antiinfectives, such as antibiotics, antimicrobials, and antiviral agents; analgesics and analgesic combinations; anorexics; antihelminthics; antiarthritics; antiasthmatic agents; anticonvulsants; antidepressants; antidiabetic agents; antidiarrheals; antiemetic and prokinetic agents; antiepileptic agents; antiestrogens; antifungal agents; antihistamines; antiinflammatory agents; antimigraine preparations; antimuscarinic. agents; antinauseants; antineoplastics; antiparasitic agents; antiparkinsonism drugs; antiplatelet agents; antiprogestins; antipruritics; antipsychotics; antipyretics; antispasmodics; anticholinergics; antithyroid agents; antitussives; azaspirodecanediones; sympathomimetics; xanthine derivatives; cardiovascular preparations, including potassium and calcium channel blockers, alpha blockers, beta blockers, and antiarrhythmics; antihypertensives; diuretics and antidiuretics; vasodilators, including general coronary, peripheral, and cerebral; central nervous system stimulants; vasoconstrictors; cough and cold preparations, including decongestants; hormones, such as estradiol and other steroids, including corticosteroids; hypnotics; immunosuppressives; muscle relaxants; parasympatholytics; psychostimulants; sedatives; tranquilizers; nicotine and acid addition salts thereof; benzodiazepines; barbituates; benzothiadiazides; beta-adrenergic agonists; beta-adrenergic antagonists; selective beta-one-adrenergic antagonists; selective beta-two-adrenergic antagonists; bile salts; agents affecting volume and composition of body fluids; butyrophenones; agents affecting calcification; catecholamines; cholinergic agonists; cholinesterase reactivators; dermatological agents; diphenylbutylpiperidines; ergot alkaloids; ganglionic blocking agents; hydantoins; agents for control of gastric acidity and treatment of peptic ulcers; hematopoictic agents; histamines; 5-hydroxytryptamine antagonists; drugs for the treatment of hyperlipiproteinemia; laxatives; methylxanthines; moncamine oxidase inhibitors; neuromuscular blocking agents; organic nitrates; pancreatic enzymes; phenothiazines; prostaglandins; retinoids; agents for spasticity and acute muscle spasms; succinimides; thioxanthines; thrombolytic agents; thyroid agents; inhibitors of tubular transport of organic compounds; drugs affecting uterine motility; vitamins; and the like; or a combination thereof.

The matrix-controlled transdermal delivery system can comprise an active component that may include, but is not limited to, flurogestone acetate, hydroxyprogesterone, hydroxyprogesterone acetate, hydroxyprogesterone caproate, medroxy-progesterone acetate, norethindrone, norethindrone acetate, norethisterone, norethynodrel, desogestrel, 3-keto desogestrel, gestadene, levonorgestrel, estradiol, estradiol benzoate, estradiol valerate, estradiol cyprionate, estradiol decanoate, estradiol acetate, ethynyl estradiol, estriol, estrone, mestranol, betamethasone, betamethasone acetate, cortisone, hydrocortisone, hydrocortisone acetate, corticosterone, fluocinolone acetonide, prednisolone, prednisone, triamcinolone, aldosterone, androsterone, testosterone, methyl testosterone, or a combination thereof.

The matrix-controlled transdermal delivery system can comprise an active component that may include, but is not limited to: a) a corticosteroid, e.g., cortisone, hydrocortisone, prednisolone, beclomethasone propionate, dexamethasone, betamethasone, flumethasone, triamcinolone, triamcinolone acetonide, fluocinolone, fluocinolone acetonide, fluocinolone acetate, clobetasol propionate, or the like, or a combination thereof; b) an analgesic anti-inflammatory agent, e.g., acetaminophen, mefenamic acid, flufenamic acid, indomethacin, diclofenac, diclofenac sodium, alclofenac, ibufenac, oxyphenbutazone, phenylbutazone, ibuprofen, flurbiprofen, ketoprofen, salicylic acid, methylsalicylate, acetylsalicylic acid, 1-menthol, camphor, slindac, tolmetin sodium, naproxen, fenbufen, or the like, or a combination thereof; c) a hypnotic sedative, e.g., phenobarbital, amobarbital, cyclobarbital, lorazepam, haloperidol, or the like, or a combination thereof; d) a tranquilizer, e.g., fulphenazine, thioridazine, diazepam, flurazepam, chlorpromazine, or the like, or a combination thereof; e) an antihypertensive, e.g., clonidine, clonidine hydrochloride, bopinidol, timolol, pindolol, propranolol, propranolol hydrochloride, bupranolol, indenolol, bucumolol, nifedipine, bunitrolol, or the like, or a combination thereof; f) a hypotensive diuretic, e.g., bendroflumethiazide, polythiazide, methylchlorthiazide, trichlormethiazide, cyclopenthiazide, benzyl hydrochlorothiazide, hydrochlorothiazide, bumetanide, or the like, or a combination thereof; g) an antibiotic, e.g., penicillin, tetracycline, oxytetracycline, metacycline, doxycycline, minocycline, fradiomycin sulfate, erythromycin, chloramphenicol, or the like, or a combination thereof; h) an anesthetic, e.g., lydocaine, benzocaine, ethylaminobenzoate, or the like, or a combination thereof; i) another analgesic, e.g., acetylsalicylic acid, choline magnesium trisalicylate, acetaminophen, ibuprofen, fenoprofen, diflusinal, naproxen and the like; j) an antipruritic agent, e.g., bisabolol, oil of chamomile, chamazulene, allantoin, D-panthenol, glycyrrhetenic acid, a corticosteroid, an antihistamines and the like; k) an antimicrobial agent, e.g., methyl hydroxybenzoate, propyl hydroxybenzoate, chlorocresol, benzalkonium chlorides, nitrofurazone, nystatin, sulfacetamide, clotriamazole, or the like, or a combination thereof; 1) an antifungal agent, e.g., pentamycin, amphotericin B, pyrrol nitrin, clotrimazole, or the like, or a combination thereof; m) a vitamin, e.g., vitamin A, ergocalciferol, cholecalciferol, octotriamine, riboflavin butyric acid ester, or the like, or a combination thereof; n) an antiepileptic, e.g., nitrazepam, meprobamate, clonazepam, or the like, or a combination thereof; o) an antihistamine, e.g., diphenhydramine hydrochloride, chlorpheniramine, diphenylimidazole, or the like, or a combination thereof; p) an antitussive, e.g., dextromethorphan, terbutaline, ephedrine, ephedrine hydrochloride, or the like, or a combination thereof; q) a sex hormone, e.g., progesterone, estradiol, estriol, estrone, or the like, or a combination thereof; r) an antidepressant, e.g., doxepin; s) a vasodilator, e.g., nitroglycerin, isosorbide nitrate, nitroglycol, pentaerythritol tetranitrate, dipyridamole, or the like, or a combination thereof; t) another drug, e.g., 5-fluorouracil, dihydroergotamine, desmopressin, digoxin, methoclopramide, domperidone, scopolamine, scopolamine hydrochloride, or the like, or a combination thereof; or the like; or a combination thereof.

In one embodiment, the transdermal delivery system is a matrix-controlled transdermal delivery system. The matrix-controlled transdermal delivery system comprises a patch evenly coated with one or more active agents directly placed on the underside of the patch. The side of the matrix-controlled transdermal delivery system coated with the one or more active agents is protected until application, for example, by a pull-off sheeting. Furthermore, the matrix-controlled transdermal delivery systems can be available in the form of a mono-coated matrix or in the form of a multi-coated matrix. Active agents include glycerol trinitrate, nicotine, and opiates and opioids, such as buprenorphine, hydromorphone, fentanyl, isofentanyl, and afentanyl.

### 6.3 Dispenser

In the present invention, the transdermal delivery system is administered by a dispensing system. The dispensing system of this invention stores and maintains the transdermal delivery system, and administers specified portions of the matrix-controlled transdermal delivery system, while preventing unauthorized access to the transdermal delivery system. The dispenser must also be able to meter the transdermal delivery system. In one embodiment, the dispenser contains a separating device for cutting the transdermal delivery system.

In one embodiment, the carriage device for the matrix-controlled transdermal delivery system and the separating device for the matrix-controlled transdermal delivery system conforms with the means normally used for such purposes and are known per se to the skilled person. U.S. Pat. No. 4,712,460 to Allen et al., the contents of which are hereby incorporated by reference, shows a drug tape dispenser and metering system. Other carriage devices known can be used in this invention including, but not limited to, reels, wheels, gears, cartridges, cassettes, and balls. Separating devices include any device that can separate, cut, or remove a length of the transdermal system from the remaining transdermal delivery system including, but not limited to, knives, blades, scissors, sharpened edges, perforators, and the like.

Specification of the dosage utilizing the ratio of dose of active agent to patch length proceeds, in the present invention, by way of a mechanical unit and/or an electronic unit. This unit serves to transpose a given desired dose into a quantitative measurement of the area of the patch to be detached or separated. With a specified width of the matrix-controlled transdermal delivery system, the area of the matrix-controlled transdermal delivery system to be separated is determined by the length thereof. For this purpose, the unit for the specification of the dosage causes the advance of a length of matrix-controlled transdermal delivery system that will provide the given dose, which then separated by the separating device of the invention. In one embodiment, the advancing of the required length is caused automatically by the electronic unit. In another embodiment, the advancing of the required length can be carried out manually, for example, by pulling the matrix-controlled transdermal delivery system out, to the length required, up to the separating point. Exemplary devices for the advancing of such materials include U.S. Pat. Nos. 6,196,740 and 5,681,123, the disclosures of which are incorporated by reference.

The conversion of the required dose into an appropriate length of the matrix-controlled transdermal delivery system can be effected electronically by the process of the invention. In another embodiment, however, the conversion of the required dosage is effected mechanically.

In one embodiment, the device of the invention comprises an electronic unit for specifying the dose, enabling a particular dose to be entered by means of a keyboard, such as a calculator-styled keyboard, whereupon an appropriate length of the matrix-controlled transdermal delivery system is electronically advanced. The length of matrix-controlled transdermal delivery system thus advanced is then detached or cut-off by the separating device of the invention.

In another embodiment, the dispenser can incorporate a mechanical unit for specifiying the dose, which transposes the required dose into an appropriate length of the matrix-controlled transdermal delivery system over two wheels that are connected to each other. The one wheel is turned until the required dosage is set and the appropriate length of the matrix-controlled transdermal delivery system to be detached is indicated on the second wheel.

The dispenser can include, in addition to or integrated with the electronic or mechanical unit, a calculating system for delivering the amount of active agent delivered over a specific period of time. The calculating system can record the entire amount of active agent consumed. This function could be optionally utilized where doses and dosage amounts must be reported to appropriate individuals or agencies, i.e. doctors, pharmacies, hospitals, administrative agencies, and the like.

Since certain active agents, such as opiates and opioids, can be subject to various laws and regulations, another embodiment of the inventive device provides additional protection from drug abuse by incorporating a locking mechanism, which can prevent the appliance from being operated. Such a locking mechanism can be activated or deactivated by an individual with the appropriate user code, number code, chip or magnet code. The locking mechanism of the present invention can be any locking mechanism that prevents the dispenser from distributing a length of the transdermal therapeutic system. These locking mechanisms include, but are not limited to, mechanical mechanisms such as keyed locks and combination locks, and electronic mechanisms, including fingerprint scans, retina scans, smart cards, key cards, electronic codes and combinations, solenoids, and the like. Exemplary mechanical locking mechanisms are disclosed by U.S. Pat. Nos. 4,936,894; 6,393,876; and 6,427,506; the disclosures of which are incorporated by reference. Exemplary electrical locking mechanisms are disclosed by U.S. Pat. Nos. 4,904984; 6,073,064; and 6,374,653; the disclosures of which are incorporated by reference. In one embodiment, the locking mechanism is an electronic mechanism that also contains user information. Such user information is then used to provide the device with information such as the length of the matrix-controlled transdermal delivery system to dispense as well as the frequency of administration. Usage information can optionally be stored in the electronic mechanism. The storage and retrieval of user information and usage information aids in the administration of the active agents by the device, as well as providing an electronic means of reporting dose and dosage administration to appropriate individuals or agencies.

In another embodiment, the device additionally incorporates a printing unit to produce a delivery slip on which the quantity of active agent dispensed from the device; the time of delivery, such as date and hour; the prescribing physician; and the user of the device, identified, for example, by the user code, are recorded.

In another embodiment, the device of the invention is designed so that throughout the entire dispensing process, including the pre-use storage of the matrix-controlled transdermal delivery system, the advancement of the matrix-controlled transdermal delivery system, detaching a length of the matrix-controlled transdermal delivery system, and optionally packaging the length of matrix-controlled transdermal delivery system, the matrix-controlled transdermal delivery system is kept under sterile conditions.

The device of the invention is suitable for use in pharmacies or hospitals, as well as the requirements of a practitioner.

Another major aspect of the present invention relates to a process for the dosing and dispensing a first length of matrix-controlled transdermal delivery systems, under sterile conditions, and incorporates the following steps:

applying a second length of matrix-controlled transdermal delivery system of a specified width and coated with at least one active agent on a carriage means;

deactivating a locking mechanism;

specifying a dose;

transposing the dose into a first length of the matrix-controlled transdermal delivery system;

separating the first length of the matrix-controlled transdermal delivery system;

optionally packaging the first length of separated matrix-controlled transdermal delivery system;

optionally providing a delivery slip, e.g., for the amount of active agent that is dispensed; the length of matrix-controlled transdermal delivery system that is dispensed, the time of delivery, and the user; and

optionally calculating the amount of active agent contained within the matrix-controlled transdermal delivery system over a specified period of time.

### 7. EXAMPLES

The following example serves to illustrate, rather than limit, the scope of the present invention.

A patch roll (matrix patch) of a width of 7 cm and a length of 70 cm is inserted into a table top device for dispensing of a length of matrix-controlled transdermal delivery systems. The matrix-controlled transdermal delivery system is evenly coated with Buprenorphine. In each 1 cm length of a matrix-controlled transdermal delivery system of a width of 7 cm, a discharge rate of 10 μg/h Buprenorphine is provided. Integral multiples of 1 cm length thus correspond to discrete steps of 10 μg/h, whereas decimal multiples correspond to steps of 1 μg/h.

Any required dosage is set by operating the keys of a keyboard. An electronic unit automatically advances the matrix patch to a length corresponding to the required dosage. The matrix patch is then separated into portions by cutting off the length of the matrix patch thus advanced. A dispensed length of 7 cm, at a width of 7 cm, corresponds to a Buprenorphine patch ordinarily obtainable on the market with a discharge rate 70 μg/h.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

All references cited are hereby incorporated herein by reference.

What is claimed is:

1. A portable device for dispensing a length of a matrix-controlled transdermal delivery system having a dose of active agent comprising:
(i) a carriage device for supporting a first length of matrix-controlled transdermal delivery system;
(ii) a unit for entering a dose amount and transposing the dose amount into a second length of the matrix-controlled transdermal delivery system;
(iii) a separating device for separating the second length of matrix-controlled transdermal delivery system from the first length of matrix-controlled transdermal delivery system; and
(iv) a locking mechanism that can be activated or deactivated by entering a user code.

2. The device of claim 1, wherein the unit for entering the dose continuously sets the dose within a specified range.

3. The device of claim 1, wherein the matrix-controlled transdermal delivery system is a patch evenly coated with the active agent.

4. The device of claim 3, wherein the active agent is directly applied to a surface of the patch.

5. The device of claim 3, wherein the active agent is combined with an adhesive or an adhesive matrix.

6. The device of claim 1, wherein the matrix-controlled transdermal delivery system is a mono-layered matrix.

7. The device of claim 1, wherein the matrix-controlled transdermal delivery system is a multi-layered matrix.

8. The device of claim 1, wherein the active agent is selected from the group consisting of glycerol trinitrate, nicotine, an opiate, an opioid, or mixtures thereof.

9. The device of claim 1, wherein the active agent is selected from the group consisting of an opiate, an opioid, and mixtures thereof.

10. The device of claim 1, wherein the unit for entering the dose is a mechanical unit.

11. The device of claim 1, wherein the unit for entering the dose is an electronic unit.

12. The device of claim 1, further comprising a printing unit for providing a delivery slip.

13. The device of claim 1, further comprising a calculating system for determining the amount of active agent dispensed over a period of time.

14. The device of claim 1, wherein the matrix-controlled transdermal delivery system is maintained and dispensed under sterile conditions.

15. A process for preparing a first length of a matrix-controlled transdermal delivery system having a dose of an active agent comprising the steps of
(i) applying a second length of matrix-controlled transdermal delivery system comprising an active agent on a carriage means;
(ii) deactivating a locking mechanism;
(iii) specifying a dose;
(iv) transposing the dose into a first length of the matrix-controlled transdermal delivery system;
(v) separating the first length of the matrix-controlled transdermal delivery system;
(vi) optionally packaging the first length of the matrix-controlled transdermal delivery system;
(vii) optionally providing a delivery slip; and
(viii) optionally calculating the amount of active agent contained within the matrix-controlled transdermal delivery system over a period of time.

16. The process of claim 15, wherein the matrix-controlled transdermal delivery system is a patch coated with the active agent.

17. The process of claim 16, wherein the active agent is applied to a surface of the patch.

18. The process of claim 16, wherein the active agent is combined with an adhesive or an adhesive matrix.

19. The process of claim 15, wherein transposing the dose into the first length of matrix-controlled transdermal delivery system is effected mechanically.

20. The process of claim 15, wherein transposing the dose into the first length of matrix-controlled transdermal delivery system is effected electronically.

21. A matrix-controlled transdermal delivery system comprising an active agent, having a length, and having markings provided at intervals of length.

22. The matrix-controlled transdermal delivery system of claim 21, wherein the markings are an imprint or a perforation.

23. The matrix-controlled transdermal delivery system of claim 22, wherein the markings can be read by a portable device for dispensing the matrix-controlled transdermal delivery system.

24. The matrix-controlled transdermal delivery system of claim 21, in the form of a patch coated with active agent.

25. The matrix-controlled transdermal delivery system of claim 24, wherein the active agent is applied to surface of the patch.

26. The matrix-controlled transdermal delivery system of claim 24, wherein the active agent is combined with an adhesive or an adhesive matrix.

27. The matrix-controlled transdermal delivery system of claim 21, in the form of a mono-layered matrix.

28. The matrix-controlled transdermal delivery system of claim 21, in the form of a multi-layered matrix.

29. The matrix-controlled transdermal delivery system of claim 21, wherein the active agent is glycerol trinitrate, nicotine, an opiate, or an opioid.

30. The matrix-controlled transdermal delivery system of claim 21, wherein the active agent is selected from the group consisting of an opiate, an opioid, and mixtures thereof.

31. The matrix-controlled transdermal delivery system of claim 29, further comprising a penetration enhancer.
